(19) United States
(12) Patent Application Publication
(10) Pub. No.: US 2024/0261593 A1
(43) Pub. Date: Aug. 8, 2024

(54) APPARATUS AND A METHOD FOR ADAPTIVE LIGHT FRACTIONATION IN PHOTOTHERAPY

(71) Applicant: Modulight Corporation, Tampere (FI)

(72) Inventors: Timo Tanila, Ylöjärvi (FI); Robert Perttilä, Tampere (FI); Petteri Uusimaa, Tampere (FI)

(73) Assignee: Modulight Corporation, Tampere (FI)

(21) Appl. No.: 18/165,645

(22) Filed: Feb. 7, 2023

(51) Int. Cl. A61N 5/067; G16H 20/40; G16H 40/63

(52) U.S. Cl. CPC A61N 5/067; G16H 20/40; G16H 40/63; A61N 2005/0626

(57) ABSTRACT

Example embodiments enable adaptive light fractionation during a biomedical illumination process, such as phototherapeutic illumination. An apparatus is configured to obtain measurements of one or more physical parameters associated with a target while an illumination program is performed on the target; determine, based on the measurements, that a predefined limit for at least one of the physical parameters is met; determine a change in at least one light fractionation setting of the illumination program when the at least one predefined limit is met; and initiate at least one action to change the at least one light fractionation setting.

### TECHNICAL FIELD

The present application generally relates to medical technology. In particular, some example embodiments of the present application relate to adaptive light fractionation for biomedical illumination systems, and in more particular, for phototherapy.

### BACKGROUND

Light fractionation settings may be selected manually based on assumptions or previous experiments performed by a user. For example, different illumination protocols may be studied in pre-clinical or clinical trials and based on averaged good results a fixed fractionation protocol is defined. However, this may be time-consuming and may not provide an optimal illumination procedure.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

Example embodiments may enable adaptive irradiance control and/or alternation between illumination and dark intervals based on measurements done continuously or sequentially during an illumination procedure either in-vitro or in-vivo.

According to a first aspect, an apparatus for adaptive light fractionation in phototherapy is provided. The apparatus comprises at least one processor; and at least one memory; the at least one memory comprising instructions which, when executed by the at least one processor, cause the apparatus at least to: obtain measurements of one or more physical parameters associated with a target while an illumination program is performed on the target; determine, based on the measurements, that a predefined limit for at least one of the physical parameters is met; determine a change in at least one light fractionation setting of the illumination program based on the at least one predefined limit that was met; and initiate at least one action to change the at least one light fractionation setting.

In an embodiment, the illumination program may be performed on the target during photodynamic therapy, direct laser therapy or laser thermotherapy.

In an embodiment, in addition or alternatively, the at least one change comprises initiation of a dark interval.

In an embodiment, in addition or alternatively, the at least one change further comprises a duration for the dark interval.

In an embodiment, in addition or alternatively, the at least one change comprises an adjustment of an irradiation level of the illumination.

In an embodiment, in addition or alternatively, the at least one action comprises sending a command to a medical device performing the illumination to change the at least one setting.

In an embodiment, in addition or alternatively, the at least one action comprises providing instructions to a user to change the setting.

In an embodiment, in addition or alternatively, the target comprises a tumor cell or tumor tissue with or without healthy cells or healthy tissue.

In an embodiment, in addition or alternatively, the measurements are obtained from measurement equipment configured to measure at least one of temperature, oxygen level, target fluorescence, drug or photosensitizer fluorescence, drug absorption, target transparency, or blood flow.

In an embodiment, in addition or alternatively, the apparatus further comprises the measurement equipment.

In an embodiment, in addition or alternatively, the at least one memory comprises instructions which, when executed by the at least one processor, cause the apparatus to store data comprising the determined changes of the light fractionation settings and corresponding measurements to the memory with information about the target; determine, based on the data, light fractionation settings for an illumination program to be performed on a similar target, the settings comprising a sequence and durations for illumination and dark intervals; and provide the light fractionation settings to at least one of a medical device or a user.

In an embodiment, in addition or alternatively, the measurements are obtained from a plurality of medical devices.

In an embodiment, in addition or alternatively, the measurements are repeated continuously.

In an embodiment, in addition or alternatively, the measurements are repeated sequentially.

In an embodiment, in addition or alternatively, the apparatus comprises a biomedical illumination device.

In an embodiment, in addition or alternatively, the apparatus comprises a phototherapeutic laser device.

According to a second aspect, a computer-implemented method for adaptive light fractionation during phototherapy is provided. The method comprises obtaining measurements of one or more physical parameters associated with a target while an illumination program is performed on the target; determining, based on the measurements, that a predefined limit for at least one of the physical parameters is met; determining a change in at least one light fractionation setting of the illumination program based on the at least one predefined limit that was met; and initiating at least one action to change the at least one light fractionation setting.

In an embodiment, the illumination program may be performed on the target during photodynamic therapy, direct laser therapy or laser thermotherapy.

In an embodiment, in addition or alternatively, the method further comprises determining optimal adaptive fractionation parameters based on combining data from multiple measurements and data sources by machine learning, neural networks or artificial intelligence algorithms.

According to a third aspect, a non-transitory computer readable medium is provided, comprising program instructions for causing that, when executed by an apparatus, cause the apparatus to perform at least the following: obtain measurements of one or more physical parameters associated with a target while an illumination program is performed on the target; determine based on the measurements, that a predefined limit for at least one of the physical parameters is met; determining a change in at least one light fractionation setting of the illumination program based on the at least one predefined limit that was met; and initiate at least one action to change the at least one light fractionation setting.

Many of the attendant features will be more readily appreciated as they become better understood by reference to the following detailed description considered in connection with the accompanying drawings.

### BRIEF DESCRIPTION OF THE DRAWINGS

The accompanying drawings, which are included to provide a further understanding of the example embodiments and constitute a part of this specification, illustrate example embodiments and together with the description help to explain the principles of the example embodiments. In the drawings:

FIG. 1 illustrates an example of an apparatus configured to practice one or more example embodiments;

FIG. 2 illustrates an example of a method for adaptive light fractionation, according to an example embodiment.

Like references are used to designate like parts in the accompanying drawings.

### DETAILED DESCRIPTION

Reference will now be made in detail to example embodiments, examples of which are illustrated in the accompanying drawings. The detailed description provided below in connection with the appended drawings is intended as a description of the present examples and is not intended to represent the only forms in which the present examples may be constructed or utilized. The description sets forth the functions of the example and a possible sequence of operations for constructing and operating the example. However, the same or equivalent functions and sequences may be accomplished by different examples.

Light fractionation may be beneficial in biomedical processes where therapeutic or illumination function may cause a change in a target environment in the biomedical process. The biomedical processes may be associated to phototherapy, for example, photodynamic therapy, direct laser therapy or laser thermotherapy. Photodynamic therapy may refer to a two-stage treatment combining light energy with a drug, such as a photosensitizer, designed to create reactive oxygen species (ROS) in the target and destroy cancerous or precancerous cells after light activation. Direct laser treatment may refer to a treatment where laser light is directly absorbed by tissue oxygen, generating ROS in the tissue without need for photosensitizer. A laser thermotherapy may refer to a treatment where tissue temperature is increased intentionally to induce cancer cell death. Laser thermotherapy may be also known as laser thermal therapy, laser interstitial thermal therapy or thermal tumor therapy. Each of these treatments may benefit from light fractionation. The biomedical processes may comprise, for example, a drug activation in an in-vitro cell line or in an ex vivo cancer tissue, in-vitro viability testing, in-vivo photodynamic therapy or direct laser therapy. The changes in a target environment may comprise, for example, a change in a temperature, a change in a content of oxygen, a change in blood flow, a change in fluorescence or a change in absorption of the drug or transparency of a tissue. Changing a light intensity level or fractionating the light on-off may help in maintaining the biomedical illumination process in more optimal process window for a targeted action, like killing cancer cells more effectively or sparing healthy cells by, for example, avoiding excessive unspecific temperature induced damage. Fractionation may also help in optimizing the illumination process for longer-term therapeutic effects like inducing immunotherapeutic antitumor effect.

Studying of a light fractionation effect to samples under study, whether in-vitro or in-vivo, can be time consuming when different fractionation settings are tested and evaluated manually. The resulted light fractionation protocols may be based on averaged results and not individually adjusted on each patient or use case. Based on the averaged results, the sample may be illuminated at a fixed irradiation level continuously or with fixed dark intervals. A light fractionation protocol may determine, for example, for how long illumination is provided on a target, for how long the illumination is shut off before continuing the illumination, and/or what is sufficient irradiation level. An illumination program may comprise settings for illumination to implement the light fractionation protocol.

An example embodiment provides adaptive light fractionation settings which may enable improved results in in-vitro and in-vivo studies or in photodynamic therapies without a need for evaluating all different light fractionation settings separately. This enables, that a treatment can be personalized to each patient and treatment session and thus improve efficacy of the treatment. The efficacy of photodynamic therapies and other phototherapeutic processes may be improved if e.g., temperature or neutral or reactive oxygen levels of a treated area are measured constantly, and adaptive light fractionation is used based on the measurements.

The adaptive light fractionation settings and the corresponding measurement results can be recorded to a cloud or other data storage for later use. For example, the same procedure can be repeated based on the recorded data in traditional devices which may not support the adaptive light fractionation and automatic changes in the light fractionation. Alternatively, the adaptive light fractionation and corresponding measurement results can be processed further by machine learning or AI (artificial intelligence) analytics and during treatment or post-treatment the processed data may be fed back to an illumination system to adapt the light fractionation further in order to improve the current or future treatments efficacy.

An adaptive light fractionation feature may be used by an apparatus configured to measure physical parameters of a target and then to automatically either adjust an irradiance to a lower level (not completely dark interval), to a higher level, or to trigger a dark interval based on the measurement. The physical parameters may comprise, for example, temperature, oxygen content, target fluorescence, drug fluorescence, photosensitizer fluorescence, tissue or drug absorption or target transparency, or other characteristics during an illumination or a dark interval. For example, irradiance of illumination or duration of dark interval may be adjusted when predefined limits of some of the characteristics are met, e.g., a predefined temperature of a target. For example, the apparatus may be configured to increase the level of irradiance when optical properties (such as transparency) of a tissue change during treatment. Hence, when the transparency is decreased to a predetermined limit, the irradiance may be increased to ensure sufficient amount of light for proper functioning of a used drug. The apparatus may be configured to decrease the level of irradiance or initiate dark interval, for example, when temperature is detected by the apparatus to increase above a predetermined limit for the temperature. The duration of dark interval or lowered/increased irradiance phase may be fixed at start time of a phase by an apparatus configured to perform the adaptive light fractionation feature. Optionally, the illumination program may be adapted to end the dark interval during the process at any time when new measurements are done and processed by the apparatus.

The adaptive light fractionation feature may be applied, for example, in a biomedical in-vitro illumination device or a phototherapeutic laser device. When the adaptive light fractionation feature is applied to a biomedical illumination device or phototherapeutic device, the traditional use of such device may not be prevented but it can be implemented as an optional feature which can be enabled or disabled. In addition, the measurements may be analysed by a deep-learning neural network model which is using measurement data recorded from many previous illumination programs. Further, control instructions for irradiance and dark intervals may be received from such system.

An objective is to find optimal light fractionation settings when using a biomedical illumination system. An apparatus may be configured for adaptive light fractionation in order to help researchers or operators to find the optimal fractionation settings, for example, when studying or treating biomedical compounds samples in-vitro. Hence, phototherapeutic device treatment efficacy may be improved. The apparatus may enable automated and/or more accurate adjustment of light fractionation settings.

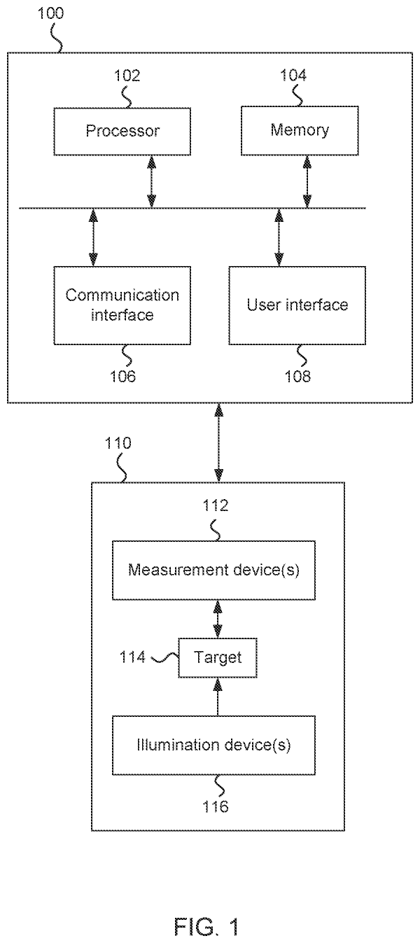

FIG. 1 illustrates an example of an apparatus 100 configured to practice one or more example embodiments.

The apparatus 100 may comprise at least one processor 102. The at least one processor 102 may comprise, for example, one or more of various processing devices, such as for example a co-processor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like.

The apparatus 100 may further comprise at least one memory 104. The memory 104 may be configured to store, for example, computer program code or the like, for example operating system software and application software. The memory 104 may be configured to store light fractionation settings for one or more medical devices, and measurement data associated with the settings. The medical devices may be configured for at least one of photodynamic therapy, direct laser therapy or laser thermotherapy. The light fractionation settings may be also stored with information about for which kind of treatment or target the light fractionation settings are for. The memory 104 may comprise one or more volatile memory devices, one or more non-volatile memory devices, and/or a combination thereof. For example, the memory 104 may be embodied as magnetic storage devices (such as hard disk drives, magnetic tapes, etc.), optical magnetic storage devices, or semiconductor memories (such as mask ROM, PROM (programmable ROM), EPROM (erasable PROM), flash ROM, RAM (random access memory), etc.).

The apparatus 100 may further comprise a communication interface 106 configured to enable the apparatus 100 to transmit and/or receive information, to/from other apparatuses. In an embodiment, the apparatus 100 may be configured to receive current light fractionation settings of a medical device. The apparatus 100 may be further configured to receive measurement data from the medical device, or separate measurement devices, during an illumination program performed by the medical device. The illumination program may refer to an illumination procedure performed by the medical device according to the light fractionation settings. The illumination program may comprise illumination of a target for a predetermined time. The illumination program may also comprise a sequence of one or more illumination intervals and one or more dark intervals during which the illumination is turned off. The apparatus 100 may be configured to send at least one of the measurement data and/or light fractionation settings determined by the apparatus 100 to one or more devices, such as a server, one or more medical devices, or an output device.

The communication interface 106 may be configured to provide at least one wireless radio connection, such as for example a 3GPP mobile broadband connection (e.g. 3G, 4G, 5G). However, the communication interface 106 may be configured to provide one or more other types of connections, for example a wireless local area network (WLAN) connection such as for example standardized by IEEE 802.11 series or Wi-Fi alliance; a short range wireless network connection such as for example a Bluetooth; a wired connection such as for example a local area network (LAN) connection, a universal serial bus (USB) connection or an optical network connection, or the like; or a wired Internet connection. The communication interface 106 may comprise, or be configured to be coupled to, at least one antenna to transmit and/or receive radio frequency signals. One or more of the various types of connections may be also implemented as separate communication interfaces, which may be coupled or configured to be coupled to a plurality of antennas.

The apparatus 100 may further comprise a user interface 108 comprising an input device and/or an output device. The input device may take various forms such a keyboard, a touch screen, or one or more embedded control buttons. The output device may comprise, for example, a display, or the like. The apparatus 100 may be configured to display data via the output device, the data comprising for example determined changes to light fractionation settings or received measurement data. An operator may be able to search for stored light fractionation settings via the input device, for example, by selecting a medical device or a target to be illuminated. The apparatus 100 may be then configured to provide, via the output device, data on the light fractionation settings based on search criteria inputted by the operator via the input device.

When the apparatus 100 is configured to implement some functionality, some component and/or components of the apparatus 100, such as for example the at least one processor 102 and/or the memory 104, may be configured to implement this functionality. Furthermore, when the at least one processor 102 is configured to implement some functionality, this functionality may be implemented using instructions comprised, for example, in the memory 104.

The functionality described herein may be performed, at least in part, by one or more computer program product components such as software components. According to an embodiment, the apparatus 100 comprises a processor 102 or processor circuitry, such as for example a microcontroller, configured by program code when executed to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), application-specific Integrated Circuits (ASICs), application-specific Standard Products (ASSPs), System-on-a-chip systems (SOCs), Complex Programmable Logic Devices (CPLDs), Graphics Processing Units (GPUs).

The apparatus 100 may be configured for adaptive light fractionation in biomedical illumination, such as adaptive light fractionation during phototherapy. The phototherapy may be implemented with one or more lasers as light source(s). The apparatus 100 may be configured to measure for example at least one of temperature, fluorescence, oxygen levels, blood flow and/or other characteristics during illumination of a target. The target may comprise a sample. The target may comprise, for example, a tumor cell or tumor tissue. The target may further comprise healthy cells or healthy tissue. The apparatus 100 may be also configured to measure at least one of the temperature, fluorescence, oxygen levels, blood flow and/or other characteristics associated with the target during a dark interval before and/or after the illumination. The measured temperature may be, for example, sample plate well specific temperature. The apparatus 100 may be configured with appropriate measuring capabilities. For example, the apparatus 100 may comprise a fluorometer or a spectrometer configured to measure various parameters of fluorescence such as intensity and wavelength distribution of emission of tissue or drug after excitation of the target. The apparatus 100 may comprise, for example, an oxygen meter, a thermometer, photodetector with or without an optical filter or a spectrometer for performing the measurements. The apparatus 100 may be configured also with other measurement equipment depending on the characteristics to be measured. Alternatively, measurements of the temperature, fluorescence, oxygen levels and/or other characteristics may be received by the apparatus 100 from one or more external measurement devices.

Based on the measurements, the apparatus 100 may be configured to automatically at least one of adjust illumination fractions, adjust an irradiance level, initiate a dark interval or end a dark interval. The adjustments and/or initiation and/or ending of the dark interval may be performed by the apparatus 100 when one or more predefined limits of one or more of the characteristics is met. In addition, or alternatively, the apparatus 100 may be configured to change one or more values of the light fractionation settings when an external system, e.g., a deep-learning neural network or machine learning model which is using the measurement data and light fractionation settings recorded from many previous illumination programs, gives applicable results on how the settings should be changed based on the measurement data. The deep-learning neural network may be trained to predict optimal light fractional settings based on obtained measurement data, and configured to provide instructions or recommendations how to change the light fractionation settings during treatment. The apparatus 100 may be configured to provide a starting point of irradiance change or dark interval and also the duration of dark interval. The duration of dark interval determined by the apparatus 100 may not be fixed but the apparatus 100 may be configured to end the dark interval at any time when new measurements are done and processed by the apparatus 100 or the external system.

For example, the apparatus 100 may be configured to control light fractionation based on temperature monitoring. The apparatus 100 may be configured to detect, for example, when temperature of a sample plate well or a target tissue has increased above a predetermined limit based on the measurements. For example, in photodynamic therapy a photochemical mechanism, which is specific to tumor cells, may be utilized. Thermal effects may be an undesired side effect and cause unspecific injury to healthy tissue surrounding the tumor. Here, fractionating the light by triggering one or more dark intervals may be helpful when the temperature increases above a predetermined limit (for example, 40° C.). The apparatus 100 may be configured to trigger a dark interval when the predetermined limit, which may depend on use case, is met. The dark interval may be triggered by sending, by the apparatus 100 to the medical device performing the illumination, a command to start the dark interval or by outputting instructions to an operator of the medical device. When the apparatus 100 then detects that the temperature has decreased below the predetermined limit, the medical device or the operator may be instructed by the apparatus 100 to continue illumination. The temperature monitoring may be also used during laser thermotherapy to adjust the temperature by light fractionation based on one or more predetermined limits for the temperature. Modification of the tissue temperature may have effect on direct thermal tumor ablation or alternatively to longer term immunotherapeutic response to therapy.

For example, the apparatus 100 may be configured to control light fractionation based on oxygen monitoring. In photodynamic therapy and direct laser therapy, oxygen may be used or be part of their mechanism to action. Hence, oxygen depletion can cause therapeutic efficacy decrease, potentially leading to undertreatment. Oxygen sensors may be configured to monitor the neutral or reactive oxygen content around an illumination target area. When the apparatus 100 obtains an indication from the oxygen sensors that oxygen concentration is decreased below a predetermined limit (for example, 3%), the apparatus 100 may be configured to trigger a dark interval. This may give time for reoxygenation of tissues between capillaries. A duration of the dark interval may depend on the target organ and level of blood flow. The apparatus 100 may be configured to cause the illumination to continue when the oxygen level is again above the predetermined limit. Alternatively, the system may monitor directly the amount of ROS in the tissue and adjust illumination according to that measurement. Another option is to monitor the level of blood flow by the apparatus 100.

For example, the apparatus 100 may be configured to control light fractionation based on drug fluorescence monitoring. In photodynamic therapy, a photoactive drug may be used that typically has intrinsic fluorescence properties. The apparatus 100 may be configured to monitor the drug fluorescence in target area. When the apparatus 100 obtains an indication that fluorescence level of the drug has decreased below a predetermined limit specific for the drug, the apparatus 100 may be configured to trigger a dark interval. When the fluorescence levels are decreased, this means that the drug may have been photobleached and no more therapeutic effect is expected. The predetermined limit for fluorescence may be, for example, a certain percentage value, an original level of the fluorescence, or detection of no fluorescence. The fluorescence may be continuously monitored by the apparatus 100, and when fluorescence signals are again detected, e.g., due to drug reperfusion to treatment site through blood vessels, the apparatus 100 may be configured to cause the illumination to continue.

The predetermined limits for one or more physical parameters may be stored on the apparatus 100 together with instructions about which changes to make on the light fractionation settings when the predetermined limit is met. The adaptive light fractionation enables changing the illumination program on the fly instead of using some fixed program which may not be optimal for the specific treatment.

The measurements may be configured to be repeated continuously or sequentially before, during and/or after an applied illumination program. The apparatus 100 may be configured to store the measurements and illumination programs. The apparatus 100 may be configured to analyse measurements and/or used illumination programs received from one or more medical devices to determine optimal light fractionation settings that can be applied on another medical device or for future treatments. Alternatively, the measurements and illumination programs may be stored to an external system, such as a cloud. The external system may comprise a deep-learning neural network or machine calculation performance and implementation available for analysis of the measurements. The external system may be configured to provide results of the measurement analysis to the apparatus 100. Alternatively, the apparatus 100 may comprise the deep-learning neural network or AI.

The results of measurement analysis may cause the apparatus 100 to trigger a change in a currently applied light fractionation. The trigger may comprise new settings for the light fractionation or a change in at least one value of the current settings. The settings may comprise duration(s) of illumination and/or dark interval(s). The settings may further comprise an intensity level of the illumination. The light fractionation may be triggered in the apparatus 100, or in another device communicatively coupled with the apparatus 100. The adaptive light fractionation procedure details may be recorded and stored to a database by the apparatus 100, and may be then reused by the apparatus 100 later on as light fractionation settings in traditional devices, and also for data analysis in external systems. The traditional device may comprise, for example, a biomedical device or a phototherapeutic device. The traditional device may be configured to use the provided settings as the light fractionation settings for new treatments to enable adaptive illumination. When the apparatus 100 comprises for example a phototherapy device, the phototherapy device may implement the measurements and other capabilities needed for adaptive light fractionation to better enable adaptive fractionation of illumination, for example, for in-vivo studies or treatments.

The apparatus 100 may comprise for example a computing device such as for example a server device, a client device, a mobile phone, a tablet computer, a laptop, or the like. Although the apparatus 100 is illustrated as a single device it is appreciated that, wherever applicable, functions of the apparatus 100 may be distributed to a plurality of devices.

In an embodiment, the apparatus 100 may comprise a medical device 110. Alternatively, the apparatus 100 may be communicatively coupled with the medical device 110. The medical device 110 may be configured for photodynamic therapy, direct laser therapy or laser thermotherapy, for example. The medical device may comprise, for example, a biomedical illumination device or a phototherapeutic laser device. The medical device 110 may comprise one or more illumination devices 116 configured for illumination of a target 114. The illumination device may comprise a light source, such as a laser. The medical device 110 may optionally comprise one or more measurement devices 112 configured to measure one or more physical parameters associated with the target 114. The medical device 110 may be configured to send measurement data received from the one or more measurement devices to the apparatus 100. The medical device 110 may be further configured to receive instructions from the apparatus 100 to adjust illumination parameters of the one or more illumination devices, such as to alter intensity or turn on or off the illumination devices.

The apparatus 100 may comprise means for performing at least one method described herein. In one example, the means comprises the at least one processor 102, the at least one memory 104 including instructions configured to, when executed by the at least one processor 102, cause the apparatus 100 to perform the method.

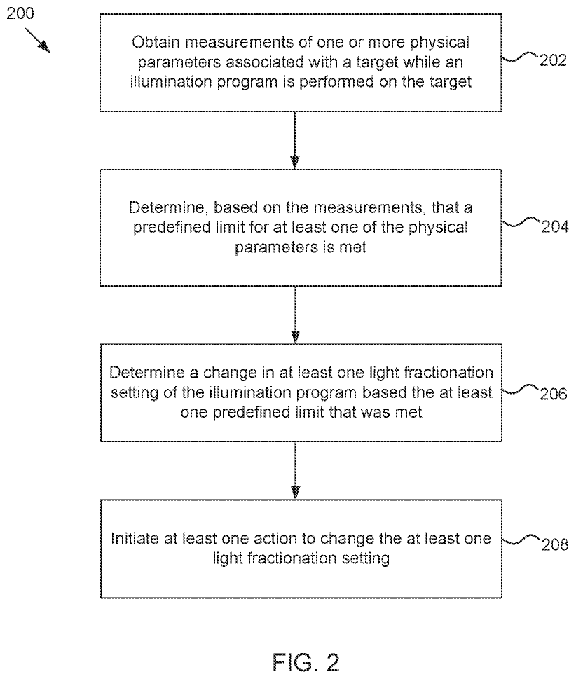

FIG. 2 illustrates an example of a method 200 for adaptive light fractionation, according to an example embodiment. The adaptive light fractionation may be performed during phototherapeutic illumination. The light fractionation may be performed, for example, during a photodynamic therapy. Alternatively, the light fractionation may be performed during a direct laser therapy. Alternatively, the light fractionation may be performed during a laser thermotherapy. The method may be implemented, for example, by the apparatus 100.

At 202, the method may comprise obtaining measurements of one or more physical parameters associated with a target while an illumination program is performed on the target.

At 204, the method may comprise determining, based on the measurements, that a predefined limit for at least one of the physical parameters is met.

At 206, the method may comprise determining a change in at least one light fractionation setting of the illumination program based on the at least one predefined limit that was met.

At 208, the method may comprise initiating at least one action to change the at least one light fractionation setting. The at least one setting may be changed during the illumination program. The change may be performed automatically. In addition, the method may comprise determining optimal adaptive fractionation parameters based on combining data from multiple measurements and data sources by machine learning, neural networks or artificial intelligence algorithms. The measurements may be received, for example, from a plurality of medical devices configured for phototherapy.

Further features of the methods directly result from the functionalities and parameters of the apparatus as described in the appended claims and throughout the specification and are therefore not repeated here. It is noted that one or more operations of the method may be performed in different order.

An apparatus may be configured to perform or cause performance of any aspect of the method(s) described herein. Further, a computer program may comprise instructions for causing, when executed, an apparatus to perform any aspect of the method(s) described herein. Further, an apparatus may comprise means for performing any aspect of the method(s) described herein. According to an example embodiment, the means comprises at least one processor, and memory including program code, the at one memory and the program code configured to, when executed by the at least one processor, cause performance of any aspect of the method(s).

Any range or device value given herein may be extended or altered without losing the effect sought. Also, any embodiment may be combined with another embodiment unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It is obvious to a person skilled in the art that with the advancement of technology, the basic idea of the present disclosure may be implemented in various ways. The present disclosure and its embodiments are thus not limited to the examples described above, instead they may vary within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The operations of the methods described herein may be carried out in any suitable order, or simultaneously where appropriate. Additionally, individual blocks may be deleted from any of the methods without departing from the scope of the subject matter described herein. Aspects of any of the embodiments described above may be combined with aspects of any of the other embodiments described to form further embodiments without losing the effect sought.

The term ‘comprising’ is used herein to mean including the method, blocks, or elements identified, but that such blocks or elements do not comprise an exclusive list and a method or apparatus may contain additional blocks or elements.

As used in this application, the term ‘circuitry’ may refer to one or more or all of the following: (a) hardware-only circuit implementations (such as implementations in only analog and/or digital circuitry) and (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions) and (c) hardware circuit(s) and or processor(s), such as a microprocessor(s) or a portion of a microprocessor(s), that requires software (e.g., firmware) for operation, but the software may not be present when it is not needed for operation. This definition of circuitry applies to all uses of this term in this application, including in any claims.

As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from scope of this specification.

1. An apparatus for adaptive light fractionation in phototherapy, the apparatus comprising:
at least one processor; and
at least one memory;
the at least one memory comprising instructions which, when executed by the at least one processor, cause the apparatus at least to:
obtain measurements of one or more physical parameters associated with a target while an illumination program is performed on the target;
determine, based on the measurements, that a predefined limit for at least one of the physical parameters is met;
determine a change in at least one light fractionation setting of the illumination program based on the at least one predefined limit that was met; and
initiate at least one action to change the at least one light fractionation setting.

2. The apparatus of claim 1, wherein the illumination program is performed on the target during photodynamic therapy, direct laser therapy or laser thermotherapy.

3. The apparatus of claim 1, wherein the at least one change comprises initiation of a dark interval.

4. The apparatus of claim 3, wherein the at least one change further comprises a duration for the dark interval.

5. The apparatus of claim 1, wherein the at least one change comprises an adjustment of an irradiation level of the illumination.

6. The apparatus of claim 1, wherein the at least one action comprises sending a command to a medical device performing the illumination to change the at least one light fractionation setting.

7. The apparatus of claim 1, wherein the at least one action comprises providing instructions to a user to change the setting.

8. The apparatus of claim 1, wherein the target comprises an in-vitro or an in-vivo sample.

9. The apparatus of claim 1, wherein the target comprises a tumor cell or tumor tissue with or without healthy cells or healthy tissue.

10. The apparatus of claim 1, wherein the measurements are obtained from measurement equipment configured to measure at least one of temperature, oxygen level, target fluorescence, drug or photosensitizer fluorescence, drug absorption, target transparency, or blood flow.

11. The apparatus of claim 10, wherein the apparatus further comprises the measurement equipment.

12. The apparatus of claim 1, wherein the at least one memory comprises instructions which, when executed by the at least one processor, cause the apparatus to:
store data comprising the determined changes of the light fractionation settings and corresponding measurements to the memory with information about the target;
determine, based on the data, light fractionation settings for an illumination program to be performed on a similar target, the settings comprising a sequence and durations for illumination and dark intervals; and
provide the light fractionation settings to at least one of a medical device or a user.

13. The apparatus of claim 12, wherein the measurements are obtained from a plurality of medical devices.

14. The apparatus of claim 1, wherein the measurements are repeated continuously.

15. The apparatus of claim 1, wherein the measurements are repeated sequentially.

16. The apparatus of claim 1, wherein the apparatus comprises a biomedical illumination device.

17. The apparatus of claim 1, wherein the apparatus comprises a phototherapeutic laser device.

18. A computer-implemented method for adaptive light fractionation during phototherapy, the method comprising:
obtaining measurements of one or more physical parameters associated with a target while an illumination program is performed on the target;
determining, based on the measurements, that a predefined limit for at least one of the physical parameters is met;
determining a change in at least one light fractionation setting of the illumination program based on the at least one predefined limit that was met; and
initiating at least one action to change the at least one light fractionation setting.

19. The method of claim 18, wherein the illumination program is performed on the target during photodynamic therapy, direct laser therapy or laser thermotherapy.

20. The method of claim 18, further comprising: determining optimal adaptive fractionation parameters based on combining data from multiple measurements and data sources by machine learning, neural networks or artificial intelligence algorithms.
